(19) United States
(12) Patent Application Publication
(10) Pub. No.: US 2018/0161562 A1
(43) Pub. Date: Jun. 14, 2018

(54) METHODS FOR COSMETIC, GALVANIC BATH-ASSISTED TRANSFER OF BIOLOGICALLY-ACTIVE SUBSTANCES

(71) Applicants: Eduard Gorenshteyn, Brooklyn, NY (US); Raisa Rozina, Brooklyn, NY (US); Stella Tokar, Brooklyn, NY (US)

(72) Inventors: Eduard Gorenshteyn, Brooklyn, NY (US); Raisa Rozina, Brooklyn, NY (US); Stella Tokar, Brooklyn, NY (US)

(21) Appl. No.: 15/788,275

(22) Filed: Oct. 19, 2017

Related U.S. Application Data
(60) Provisional application No. 62/432,072, filed on Dec. 9, 2016.

(51) Int. Cl. A61M 37/00; A61K 8/73; A61K 8/65; A61K 8/64; A61Q 19/08; A61N 1/32

(52) U.S. Cl. CPC A61M 37/00; A61K 8/735; A61K 8/65; A61K 8/64; A61Q 19/08; A61F 7/00; A61N 1/325; A61K 2800/83; A61K 2800/74; A61M 2037/0007; A61N 1/327

(57) ABSTRACT

Methods are described for providing a cosmetic procedure to deliver biologically-active substances, such as hyaluronic acid, collagen, and elastin to dermal, intradermal, and subdermal regions of the skin in a safe and efficient manner. In an implementation, a method embodiment includes, but is not limited to, introducing an aqueous cosmetic solution to a galvanic bath, the aqueous cosmetic solution including one or more biologically-active substances; introducing an individual subject to the galvanic bath; and applying an electrical current via the galvanic bath to the aqueous cosmetic solution to introduce the one or more biologically-active substances to a skin surface of the individual subject.

### CROSS-REFERENCE TO RELATED APPLICATIONS

The present application claims the benefit of 35 U.S.C. § 119(e) of U.S. Provisional Application Ser. No. 62/432,072, filed Dec. 9, 2016, and titled “METHODS FOR COSMETIC, GALVANIC BATH-ASSISTED TRANSFER OF BIOLOGICALLY-ACTIVE SUBSTANCES.” U.S. Provisional Application Ser. No. 62/432,072 is herein incorporated by reference in its entirety.

### BACKGROUND

Cosmetic skin care products and treatments strive to maintain a healthy and youthful skin appearance, with focus on elasticity, structure, smoothness, and coloration. Such products and treatments can provide localized effects, such as through topical creams and ointments, oral medications or supplements, or needle-based injections, which attempt to reverse or slow damaging effects to the skin caused by sun exposure or overexposure, aging, or diet.

### SUMMARY

Methods are described for a cosmetic process to deliver biologically-active substances, such as hyaluronic acid, collagen, and elastin to dermal, intradermal, and subdermal regions of the skin in a safe and efficient manner. In an implementation, a method embodiment includes, but is not limited to, introducing an aqueous cosmetic solution to a galvanic bath, the aqueous cosmetic solution including one or more biologically-active substances; introducing an individual subject to the galvanic bath; and applying an electrical current via the galvanic bath to the aqueous cosmetic solution to introduce the one or more biologically-active substances to a skin surface of the individual subject.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. The use of the same reference numbers in different instances in the description and the figures may indicate similar or identical items.

FIG. 1 is a flow chart of a cosmetic method using galvanic bath-assisted transfer of biologically-active substances in accordance with example implementations of the present disclosure.

### DETAILED DESCRIPTION

### Overview

An individual's skin appearance is generally dependent on three main components: hyaluronic acid (e.g., to aid in retaining water in the skin, tissue repair, etc.), collagen (e.g., to provide structural support to the skin), and elastin (e.g., to keep skin smooth, elastic, etc.). At birth, an individual has approximately fifteen grams of hyaluronic acid present in their body, with about half concentrated in the skin. Beginning at about twelve years of age, and due to multiple intrinsic and extrinsic factors (including, but not limited to, aging, sun exposure, sun overexposure, sun burns, etc., or combinations thereof), the skin begins to lose hyaluronic acid. For instance, by about thirty years of age, an individual may have approximately half of the content of hyaluronic acid (e.g., 7.5 g in the body). This reduction in hyaluronic acid can negatively affect water retention by the skin, resulting in drier skin, looser skin, or an otherwise aged appearance.

Cosmetic skin care products and treatments, such as topical creams and ointments or needle-based injections, are used in an attempt to reverse or slow damaging effects to the skin caused by sun exposure or overexposure, aging, or diet. However, such products and treatments have limited applications, cause undesirable side effects, or can be onerous in use. For example, oral supplements of hyaluronic acid, collagen, and elastin can be difficult for a body to maintain, due in part to deactivation of the base components (e.g., proteins) caused by pH and digestive enzymes upon passage through the gastrointestinal tract. Topical creams, ointments, and the like can have limited penetration of the skin, caused in part by a low mobility of certain components (e.g., oil-based components) to penetrate superficial corneal layers of skin, which can include dead or damage skin cells. Further, such applications can require a strict regimen of use, such as daily applications. Localized patches (e.g., passive transport patches) can be used to deliver skin care products to the skin, however such patches can rely on ineffective photokinesis methods of delivery, and can require constant follow-up applications. Active patches can use electrodes mounted to an individual's skin to provide stimulus to assist in the penetration of particular skin products, however the direct contact can cause irritation, or can burn or otherwise damage the skin. Such electrodes can include, but are not limited to, galvanic electrodes, ultrasound electrodes, electrical electrodes, photophoretic electrodes, or the like. Needle-based injections can be used for transdermal delivery of skin care products such as injectable fillers (e.g., RESTYLANE™), neurotoxic proteins (e.g., BOTOX™), or the like. The needle-based injections can involve painful applications of the skin care product, can cause irritation at the injection site(s), can involve expensive procedures, can require a licensed physician to perform the invasive procedure, can result in complications such as hematomas or local or general infections, which in turn can require further medical therapy with antibiotics, steroids, or in certain circumstances, require hospitalization or surgery.

Accordingly, the present disclosure is directed to methods for providing a cosmetic procedure to deliver biologically-active substances, such as hyaluronic acid, collagen, and elastin to dermal, intradermal, and subdermal regions of the skin in a safe and efficient manner. Such treatments can include, for example, spa treatments, hydrotherapy treatments, or the like. In implementations, the biologically-active substances are actively transferred from a galvanic bath to and through the skin via at least one of iontophoresis, ionophoresis, or electrophoresis. The galvanic bath provides a total body application of the biologically-active substances to the skin (i.e., not limited to a local application), where such application can be provided simultaneously to and through all exposed skin regions within the galvanic bath, without damage to the cellular surface, tissue, or membrane.

### Example Implementations

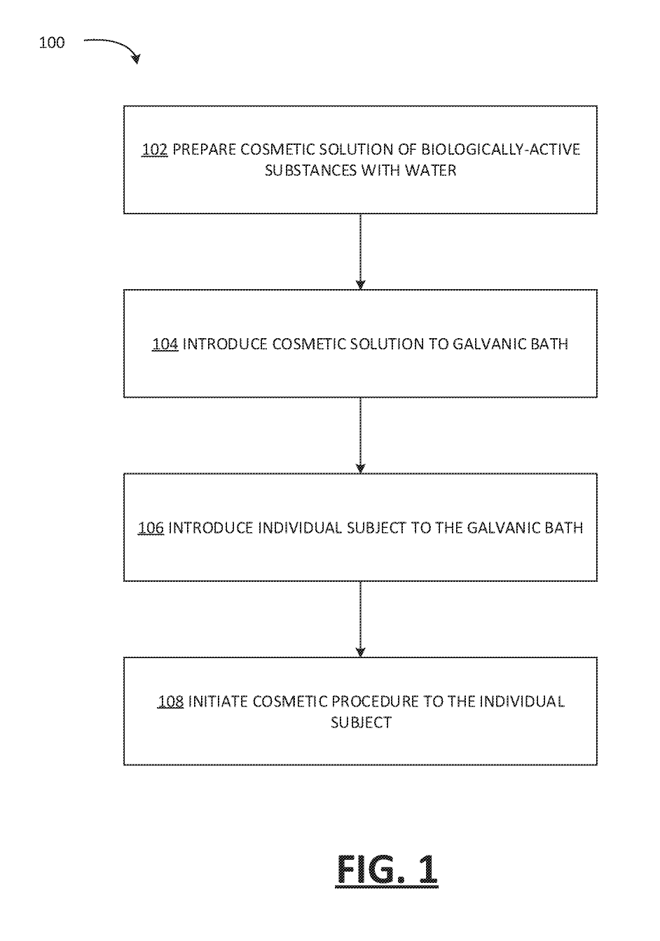

Referring generally to FIG. 1, a method 100 for a cosmetic procedure using galvanic bath-assisted transfer (e.g., iontophoresis, ionophoresis, electrophoresis, combinations thereof) of biologically-active substances is provided in accordance with example implementations of the present disclosure. The method 100 includes preparing a cosmetic solution (or mixture, colloid, etc.) of biologically-active substances in water in block 102. The biologically-active substances can include, but are not limited to, hyaluronic acid (a glycosaminoglycan), collagen, elastin, and combinations thereof. The biologically-active substances can have a molecular weight of about 20 kDa to about 2,500 kDa. In an implementation, the collagen and elastin are proteins with an average molecular weight of about 500 kDa to about 600 kDa. The cosmetic solution can also include materials including, but not limited to, vitamins, hormones, antioxidants, cosmetic agents, and the like, where such materials can be water soluble. The cosmetic solution can be prepared with water to provide a concentration of water soluble biologically-active substances in water of about 0.025 percent to about 1 percent by weight. The water provides a medium through which electric current from a galvanic bath can pass to facilitate active transfer (e.g., iontophoresis, ionophoresis, electrophoresis, combinations thereof) of the biologically-active substances through dermal, intradermal, and subdermal layers of the skin.

The method 100 also includes introducing the cosmetic solution to a galvanic bath in block 104. In an implementation, the preparing the cosmetic solution of biologically-active substances in water (e.g., block 102) can be accomplished directly in the galvanic bath. In an alternative implementation, a pre-mixed cosmetic solution (or mixture, colloid, etc.) is added to the galvanic bath. The galvanic bath can have a volume suitable to immerse the full body (or portions thereof, such as the face) of an individual subject for interacting with the total body skin area (e.g., simultaneously, substantially simultaneously, etc.). The full body immersion can include exclude the head of the individual or can include the head of the individual. The galvanic bath is configured to deliver the cosmetic solution to an individual subject in the galvanic bath via active transfer (e.g., iontophoresis, ionophoresis, electrophoresis, combinations thereof) of the biologically-active substances through dermal, intradermal, and subdermal layers of the skin, such as via high frequency, low current application of current through the water in the galvanic bath. In an implementation, the galvanic bath is configured to apply an electrical current having a frequency of about 500 Hz to about 600 Hz, which can be varied throughout a cosmetic procedure. For instance, a frequency of about 600 Hz can facilitate activate transfer of the biologically-active substances to dermal and/or intradermal regions, and a frequency of about 500 Hz can facilitate activate transfer of the biologically-active substances to subdermal regions. In an implementation, the galvanic bath is configured to apply an electrical current at about 300 mA to about 500 mA, where such application can be provided as a pulsed application. In an implementation, the galvanic bath is configured to modify a waveform shape of the electrical current applied to the cosmetic solution. For example, the galvanic bath can generate the electrical current with a monophasic sinusoidal waveform shape for superficial skin application (e.g., for dermal and/or intradermal application), and can generate the electrical current with a monophasic square waveform shape for transdermal skin application (e.g., active pumping of the biologically-active substances to subdermal layers of the individual). The galvanic bath can include a heating element configured to modify or maintain a temperature of the cosmetic solution, such as to maintain a constant solution temperature, to provide a pattern or gradient of heating during application, or the like. In an implementation, the galvanic bath maintains the cosmetic solution at a temperature of about 36° C.

The method 100 also includes introducing an individual subject to the galvanic bath in block 106. As described herein, the galvanic bath provides for full body immersion of the individual. The individual can enter the galvanic bath having a volume of water suitable for full body immersion, or can enter the galvanic bath having less water, where the water is introduced to the galvanic bath until a suitable volume is present. In an implementation, the individual is not fitted with any electrodes used by the galvanic bath to generate the electrical current. For example, any electrodes used by the galvanic bath to generate the electrical current directly interact with the cosmetic solution and not with the individual (e.g., no physical contact between the individual subject and the electrode(s)).

The method 100 also includes initiating the cosmetic procedure to the individual subject in block 108. For instance, the galvanic bath can direct the electrical current according to one or more cosmetic protocols to active transfer (e.g., iontophoresis, ionophoresis, electrophoresis, combinations thereof) of the biologically-active substances through dermal, intradermal, and subdermal layers of the skin. As an example, the galvanic bath can direct the electrical current according to a first cosmetic protocol, whereby a particular frequency, current, waveform, pulse pattern, or combinations thereof can be provided to and through the cosmetic solution for a given time. For example, the galvanic bath can provide focused application to the superficial dermal layer and/or intradermal layer through a first cosmetic protocol of a 600 Hz frequency, an electrical current at about 300 mA to about 500 mA, a monophasic sinusoidal waveform shape, or the like. The galvanic bath can then direct the electrical current according to a second cosmetic protocol, whereby one or more of the frequency, current, waveform, or pulse pattern can differ from the first cosmetic protocol for a given time, such as by providing focused application to subdermal layers through a second cosmetic protocol of a 500 Hz frequency, an electrical current at about 300 mA to about 500 mA, a monophasic square waveform shape, or the like. These cosmetic protocols are examples, and do not limit the scope of the available cosmetic options of the galvanic bath. For instance, the galvanic bath can direct the characteristics of the electrical current applied to and through the cosmetic solution according to any number of cosmetic protocols, such as by controlling or modifying a current type (e.g., alternating current, direct current), a current magnitude, a current frequency, a shape of current pulse, a time of current generation, or combinations thereof. In an implementation, the individual subject remains immersed within the galvanic bath for a duration of about ten minute to about twenty minutes.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

1. A method for a cosmetic skin procedure, comprising:
introducing an aqueous cosmetic solution to a galvanic bath, the aqueous cosmetic solution
including one or more biologically-active substances, the one or more biologically-active substances including at least one of hyaluronic acid, collagen, or elastin;
introducing an individual subject to the galvanic bath; and
applying an electrical current via the galvanic bath to the aqueous cosmetic solution to introduce the one or more biologically-active substances to a skin surface of the individual subject.

2. The method of claim 1, wherein the one or more biologically-active substances include hyaluronic acid.

3. The method of claim 1, wherein the one or more biologically-active substances include collagen.

4. The method of claim 1, wherein the one or more biologically-active substances include elastin.

5. (canceled)

6. The method of claim 1, wherein the one or more biologically-active substances include each of hyaluronic acid, collagen, and elastin.

7. The method of claim 1, wherein introducing the individual subject to the galvanic bath includes immersing the full body of the individual subject in the aqueous cosmetic solution.

8. The method of claim 1, wherein applying the electrical current via the galvanic bath to the aqueous cosmetic solution to introduce the one or more biologically-active substances to the skin surface of the individual subject includes applying the electrical current via the galvanic bath to the aqueous cosmetic solution to introduce the one or more biologically-active substances to the skin surface of the individual subject without contact between the skin surface of the individual subject and an electrode of the galvanic bath.

9. The method of claim 1, wherein applying the electrical current via the galvanic bath to the aqueous cosmetic solution to introduce the one or more biologically-active substances to the skin surface of the individual subject includes applying the electrical current via the galvanic bath according to at least a first cosmetic protocol and a second cosmetic protocol, the second cosmetic protocol differing from the first cosmetic protocol according to one or more of a current type, a current magnitude, a current frequency, a shape of current pulse, or a time of current generation.

10. A method for a cosmetic skin procedure, comprising:
introducing an aqueous cosmetic solution to a galvanic bath, the aqueous cosmetic solution including one or more biologically-active substances;
introducing an individual subject to the galvanic bath; and
applying an electrical current via the galvanic bath to the aqueous cosmetic solution to introduce the one or more biologically-active substances to a skin surface of the individual subject, the electrical current having a frequency of about 500 Hz to about 600 Hz.

11. The method of claim 10, wherein applying the electrical current via the galvanic bath to the aqueous cosmetic solution to introduce the one or more biologically-active substances to the skin surface of the individual subject includes applying the electrical current at about 300 mA to about 500 mA via the galvanic bath to the aqueous cosmetic solution to introduce the one or more biologically-active substances to the skin surface of the individual subject.
